(19) United States
(12) Patent Application Publication
(10) Pub. No.: US 2024/0335198 A1
(43) Pub. Date: Oct. 10, 2024

(54) VASCULAR ANASTOMOTIC COUPLING DEVICE

(71) Applicant: BOARD OF TRUSTEES OF SOUTHERN ILLINOIS UNIVERSITY, Carbondale, IL (US)

(72) Inventors: Michael W. Neumeister, Springfield, IL (US); Jessie L. Koljonen, Springfield, IL (US); John C. Johnson, Phoenix, AZ (US)

(21) Appl. No.: 18/616,545

(22) Filed: Mar. 26, 2024

Related U.S. Application Data
(60) Provisional application No. 63/494,324, filed on Apr. 5, 2023.

(51) Int. Cl. A61B 17/11; A61B 17/00

(52) U.S. Cl. CPC A61B 17/11; A61B 2017/00477; A61B 2017/00862; A61B 2017/1107; A61B 2017/1121; A61B 2017/1132

(57) ABSTRACT

A vascular anastomotic coupling device includes, for each of two vessels prepared for anastomosis, an inner ring sized to slide over an outer diameter of one of the two vessels. An outer ring is configured to trap an everted portion of the one of the two vessels between itself and the inner ring. Interface features on the inner ring and outer ring are configured to trap and hold the everted portion of the one of the two vessels between them and fix a position of the inner ring and outer ring with respect to each other. A connection interface on the outer ring is configured, when mounted on the one of the two vessels, to join with the outer ring of the other of the two vessels to join the two vessels and complete anastomosis of the two vessels.

### PRIORITY CLAIM AND REFERENCE TO RELATED APPLICATION

The application claims priority under 35 U.S.C. § 119 and all applicable statutes and treaties from prior U.S. provisional application Ser. No. 63/494,324, which was filed Apr. 5, 2023.

### FIELD

A field of the invention is surgical instruments. The invention is generally applicable to join two vessel ends, or other tubular organs to accomplish anastomosis. A particular application of the invention is to join veins and arteries.

### BACKGROUND

Current common surgical instruments for vein anastomoses in microsurgery are ring pin coupler devices. With these instruments, the ends of two veins are folded over pins on separate rings, and the two rings are brought together to create the anastomosis. FIGS. 1A-1D show the basic structure and operation of such a device. FIG. 1A shows a perspective view of the ring pin coupler in a coupler device, FIG. 1B shows the ring ping coupler, FIG. 1C shows separate halves of the ring pin coupler attached to separate ends of a vessel and held in the coupler device, and FIG. 1D shows the ring ping coupler in its installed state to reconnect ends of a vessel. A commercially available ring pin coupler device applicable to arterial coupling is a vessel everter offered by GEM Synovis and referred to as the GEM Flow Coupler.

Despite the availability of the GEM flow coupler, arteries are still often hand-sewn together by surgeons. Arteries have thicker and more muscular walls than veins. With respect to ring pin coupling devices, this can make it quite challenging to spread the vessel over the ring-pin device. Surgeons often find it frustrating that the arterial wall pops off one anchoring pin as the vessel is attached to the next pin. Hand sewing requires great skill and carries inherent risks. Often, there is significant time required to prepare the vessel ends to ensure the stitch does not catch adventitia (outer tissue) within the vessel, with further time required to hand-stitch the circumference of the artery. In microsurgery, time is critical to tissue survival. Once the blood supply to the artery is clamped off to perform the anastomosis, it is critical to minimize the ischemia (no blood flow through the artery) time. Prolonged ischemia times could lead to loss of the vessel flap and failure of the surgery.

Examples of ring pin coupler devices are described in the following publications: Berger et al., U.S. Pat. No. 4,607,637; Plott et al., WO 2017/040884; and Busch et al., WO 2020/153954. Knight et al., U.S. Pat. No. 6,575,985 describes an instrument for everting the end of a vessel. A conical holder and mandrel are used to evert a vessel end, which can then be sutured. The instrument is described as less traumatic to the intima of a vessel during the eversion procedure than conventional surgical graspers. However, the need to conduct suturing carries the risk of too long of a period of ischemia, as discussed in the previous paragraph.

### SUMMARY OF THE INVENTION

A preferred embodiment provides a vascular anastomotic coupling device that includes, for each of two vessels prepared for anastomosis, an inner ring sized to slide over an outer diameter of one of the two vessels. An outer ring is configured to trap an everted portion of the one of the two vessels between itself and the inner ring. Interface features on the inner ring and outer ring are configured to trap and hold the everted portion of the one of the two vessels between them and fix a position of the inner ring and outer ring with respect to each other. A connection interface on the outer ring is configured, when mounted on the one of the two vessels, to join with the outer ring of the other of the two vessels to join the two vessels and complete anastomosis of the two vessels. The connection interface can be formed, for example, from outward facing pins, or from magnets, a mechanical capture, adhesive or suture openings. The anastomotic coupling device can be completely abluminal after complete anastomosis, and no portion of the coupling device penetrates the vessels.

A preferred anastomosis implantation device for implantation of the preferred vascular anastomotic coupling device includes a vessel support mounted on a guide wire and sized to fit within the vessel. A flaring tool is configured to slide along the wire guide, carry an outer ring, and flare a portion of the vessel such that the outer ring can evert the portion over the inner ring.

### BRIEF DESCRIPTION OF THE DRAWINGS

FIGS. 1A-1D (Prior Art) are perspective views of a known ring pin coupler and coupler device and the basic method used to apply the ring pin coupler to reconnect a vessel;

FIGS. 2A-2B are perspective views of a preferred vascular anastomotic coupling device during the eversion/attachment implantation part of the procedure (FIG. 2A) and then during the anastomosis procedure (FIG. 2B) of joining two everted vessel portions by connecting the outer rings of the coupler device; and

FIGS. 3A-3H are schematic cross-sectional views of steps used to employ a a preferred embodiment vascular anastomotic coupling device to rejoin ends of a vessel.

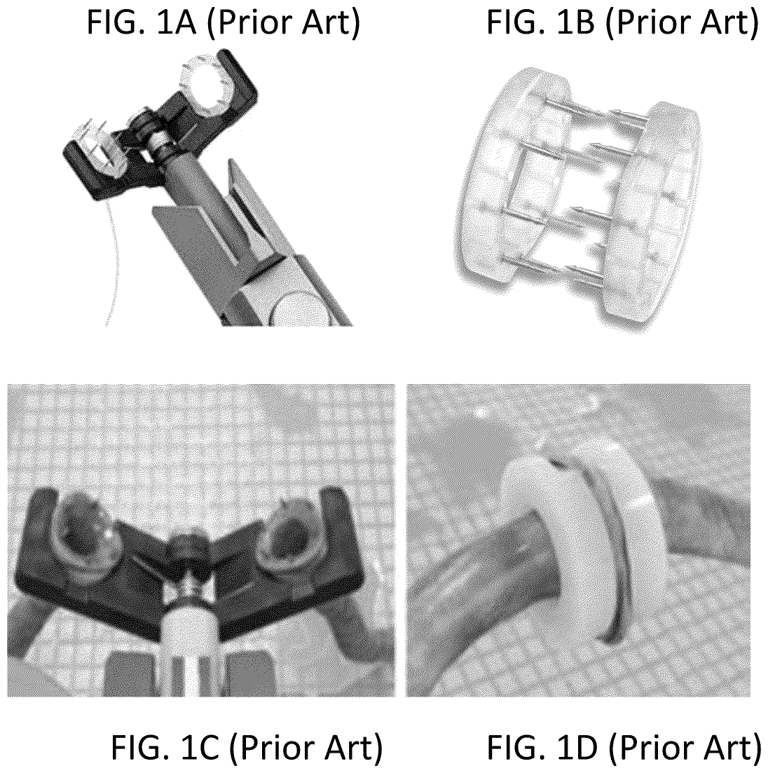

### DETAILED DESCRIPTION OF THE PREFERRED EMBODIMENTS

Preferred embodiments provide a vascular anastomotic coupling device that requires no pins to penetrate a vessel, provides fast and secure coupling of vessel ends, and can be installed very quickly to avoid extended ischemia time. The device can be installed without any penetration or disturbance of the inner wall of the vessel. The device includes inner and outer rings that trap an everted portion of a vessel end between them, and outer rings that connect to each other to complete anastomosis of two vessels.

A preferred anastomosis implantation device is configured to implant the preferred vascular anastomotic coupling device. The inner rings are configured to mount on a vessel, and the outer rings can be installed on an eversion flaring tool of the anastomosis implantation device. A conical outer surface everts the vessel end while advancement of the outer ring folds the vessel end over the inner ring, with a vessel support sized to enter the vessel.

After the inner ring is attached, the flaring tool can be slid along a common guide wire that was used to place the vessel support. The flaring tool is advanced along the guide wire until the outer ring interfaces into a fixed position over the inner ring with the everted portion of the vessel therebetween. Advancement of the flaring tool initially causes the vessel to fold over the inner ring due to the conical surface of the flaring tool. An outer ring carried by the flaring tool can be used to push the flared vessel portion back over the inner ring.

Implantation into the fixed position can be accomplished, for example, via interface features such as an inner circumferential set of tabs or a ridge on the outer ring that mate with a circumferential groove on the inner ring with a portion of the everted vessel between the ridge and groove. With the outer ring being slightly expanded when mounted, the flaring tool and vessel support can be axially removed to leave the end of the vessel fully prepared with an everted portion locked between the inner and outer rings. Flexible material is preferred for at least the outer ring but is not required. During installation, the vessel can be slightly compressed, which provides some flexibility to the system of the inner ring, outer ring and the vessel itself. Some ring interface features don't require flexibility, for example the rings can be configured with a complementary inner/outer gear tooth surface. Once the outer ring is pushed over the inner ring, it is rotated so the gear teeth interfere with each other and the ring cannot back out.

A second vessel end is prepared in the same manner with inner and outer rings, then anastomosis is accomplished by connecting the outer rings of the vessel ends.

Such connection between the outer rings can be accomplished, for example, by mechanisms including pins, tabs, tongue and groove, or snap or friction fittings. The connection of the outer rings to each other can be direct without any vessel portion between the interface of the outer rings to each other. Other connection techniques include magnets, mechanical capture (clips, slotting, compression, interference), UV cured medical adhesive, etc. The outer rings can also include openings for suturing the outer rings together. Additional examples that are used in other vascular implant devices can also be used for the connection interface and include a spring clip, mechanical capture using compressible features in ring mating surfaces, protruding pins on the inner ring that pierce and capture the outer ring or vice versa, locking features in the outer ring engaged by limited relative rotation of the inner and outer ring, an adhesive applied to one or both of the inner and outer ring, suture holes in the inner and outer rings, and complementary hooks and posts in the inner and outer rings.

Preferred vascular anastomotic coupling devices can provide reliable, quick, and simple connection of veins, arteries and other vessels/body lumens. Advantageously, there is minimal vessel preparation required. Once installed, devices of the invention ensure that no material of the coupler is inside the joined vessels, e.g., the coupler is completely abluminal with respect to the two joined vessels.

Preferred anastomosis implantation devices provide a reliable but simple method of installation that can reduce tedious, error-prone steps that can affect the reliability of the anastomosis. When used to provide anastomosis for an artery, there is no requirement for a surgeon or technician to manually pull a thick vessel wall of an artery over numerous tabs. Compared to a traditional hand-sewn technique, the mounted inner and outer rings of the present device themselves keep the vessels open, reducing the chance of stenosis (narrowing) and failure of the anastomosis due to occlusion. Less time and surgical skill are needed, leading to a higher likelihood of a successful outcome that is both reproducible and reliable.

Preferred embodiments of the invention will now be discussed with respect to experiments and drawings. Broader aspects of the invention will be understood by artisans in view of the general knowledge in the art and the description of the experiments that follows.

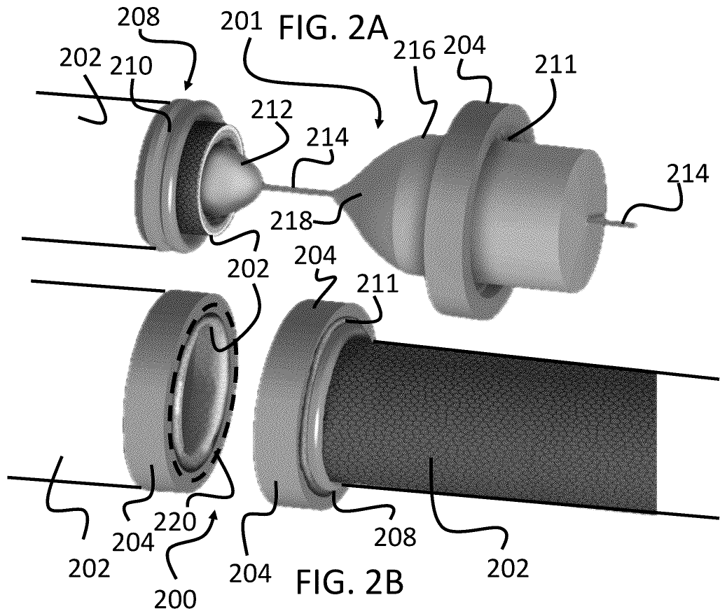

FIGS. 2A-2B illustrate a preferred vascular anastomotic coupling device 200 during an eversion/attachment implantation part of the procedure (FIG. 2A) with a preferred anastomosis implantation device 201 and then during an anastomosis procedure (FIG. 2B) of joining two everted portions of a vessel 202 by connecting outer rings 204 of the coupler device 202. On each vessel end is mounted an inner ring 208, which is sized according to the outer diameter of the vessel 202.

Interface features are included on the inner ring 208 and outer ring 204 configured to trap and hold an everted portion of the one of the vessels 202 between them and fix a position of the inner ring 208 and outer ring 204 with respect to each other. A preferred interface feature is a groove and ridge arrangement. The inner ring 208 can be a single ring with a circumferential groove 210 or the inner ring 208 can include multiple rings such that the groove 210 is formed between them. The groove 210 is a preferred structure to fix position of an outer ring 204 that has a complementary ridge 211 onto the inner ring 208 with a terminal and everted portion of an end of the vessel 202 trapped between.

This procedure is illustrated in FIG. 2A, where a vessel support 212 of the anastomosis implantation device 201 is on a guide wire 214. The vessel support 212 is sized to fit within the vessel 202 and configured to support the vessel 202 and hold it open during an implantation procedure. A flaring tool 216 of the anastomosis implantation device 201 is configured with an inner lumen to slide along the guide wire 214 and carry an outer ring 204. A tapered, conical end 218 of the flaring tool 216 is configured to flare a terminal end portion of the vessel 202 such that the outer ring 204 can evert the portion over the inner ring 208 as the flared portion moves into the vessel 202 causing eversion with the outer ring 204 to allow trapping of the vessel portion between the outer ring 204 and the inner ring 208. The inner ring 208 and and outer ring 204 include the interface features configured to trap and hold the everted vessel portion between them and fix a position of the inner ring 208 and outer ring 204 with respect to each other. With the illustrated connection interface, the outer ring 204 has the circular ridge 211 that presses the everted portion of the vessel 202 into the circumferential groove 210 of the inner ring(s) 208. The ridge 211 and/or the outer ring 204 are preferably flexible so to expand slightly when it traps the vessel portion between it and the inner ring 208.

Each of two vessel ends is prepared with the everted portion of the vessel end between an outer ring 204 and inner ring 208. The outer rings 204 on separate vessel ends each include a connection interface 220 to join to each other to complete anastomosis of respective donor and recipient vessels that have an installed inner 208 and outer ring 204.

Opposing surfaces of the outer rings 204 include the connection interface 220, which can be magnets, a mechanical capture (clips, slotting, compression, interference, pin/hole), UV cured medical adhesive or openings for suturing the outer rings together.

Other interface features between an inner circumference of the outer ring 204 and outer circumference of the inner ring 208 to trap the everted portion of the vessel can be, for example a spring clip, mechanical capture using compressible features in ring mating surfaces, protruding pins on the inner ring that pierce and capture the outer ring or vice versa, locking features in the outer ring engaged by limited relative rotation of the inner and outer ring, an adhesive applied to one or both of the inner and outer ring, suture holes in the inner and outer rings, and complementary hooks and posts in the inner and outer rings.

Materials for the inner 208 and outer 204 rings and the interface features between them must be biocompatible and can be made from a variety of materials including biocompatible polymers, metal and alloys. While the material(s) must be biocompatible, unlike many implanted devices, the coupler 200 does not need to form an adhesion or bond with the tissue, bone, or cartilage. Clotting is not a concern as the coupler 200 does not have exposure to the interior of the blood vessel 202. There is therefore no risk of the material or the shape of the coupler causing biological material to accumulate on its surface. After implantation, the coupler 200 will see very little mechanical stress once installed. The material can also be bioerodible. As the joined vessels heal together, so the coupler can then dissolve over time.

Example materials suitable for the inner 208 and outer 204 rings include commonly implanted metals and alloys (stainless steel, cobalt-chrome alloy, titanium, nickel-titanium alloy (nitinol), platinum, silver, iridium, tungsten, etc.). Preferred materials for the inner 208 and outer 204 rings include UHMW (ultra-high molecular weight) polyethylene, and plastics/polymers with sufficient mechanical strength to permit the implantation procedure.

The sizing of the inner rings 208 is slightly larger than the outer diameter of a vessel targeted for implantation/anastomosis. This can vary somewhat from patient to patient, and a preferred implantation surgical kit includes various sized ring kits, e.g., ranging with an inner ring diameter from 0.8 mm to about 2 or 3 cm. The coupler 200 is completely abluminal to the vessel 202 and no part of the coupler penetrate the vessel (vein or artery). The thickness of the vessels is not an impediment to implantation of the present coupler 200 and larger vessels can be repaired via implantation/anastomosis of a present coupler device as compared to the conventional ring pin coupler devices discussed in the background.

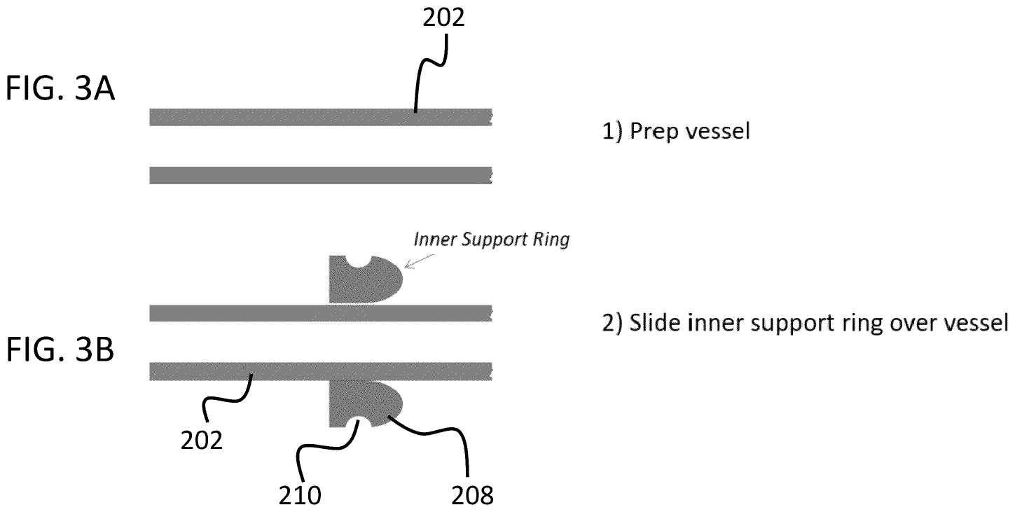

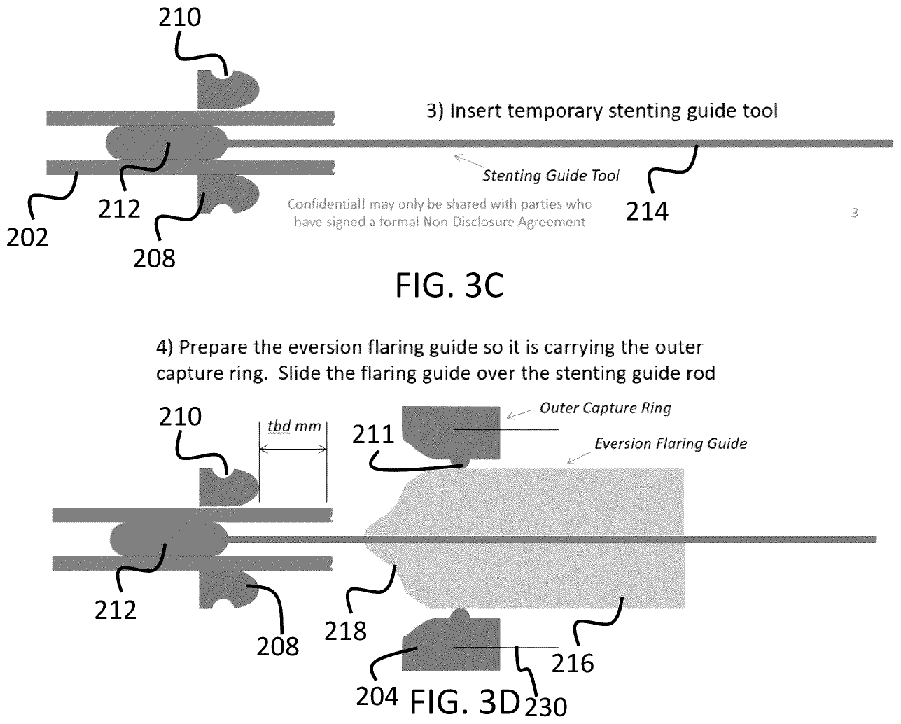

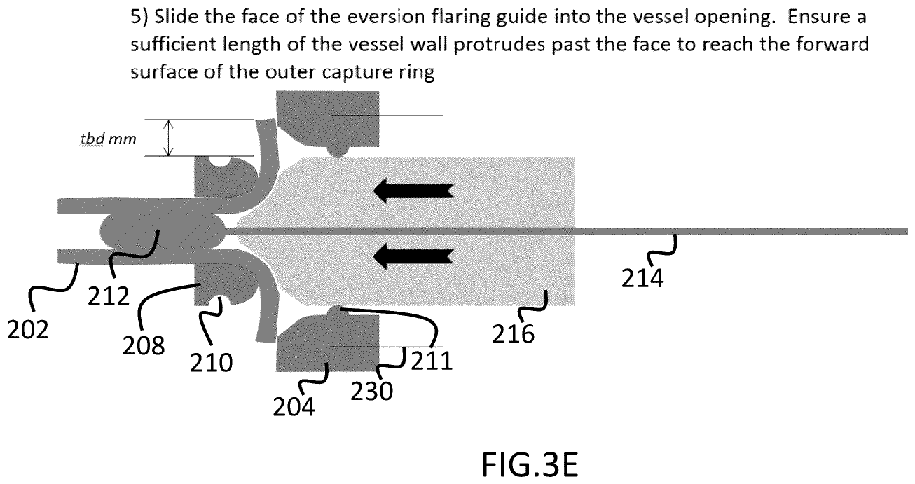

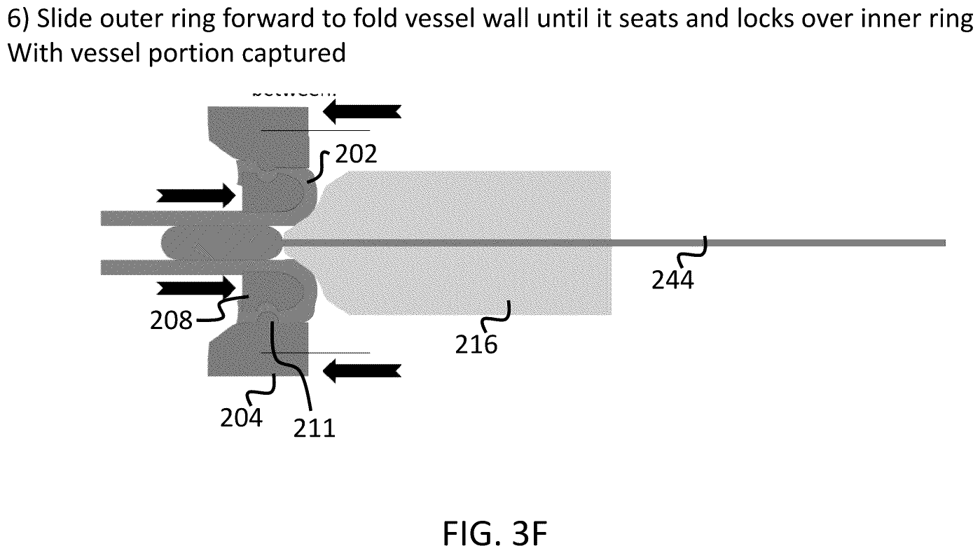

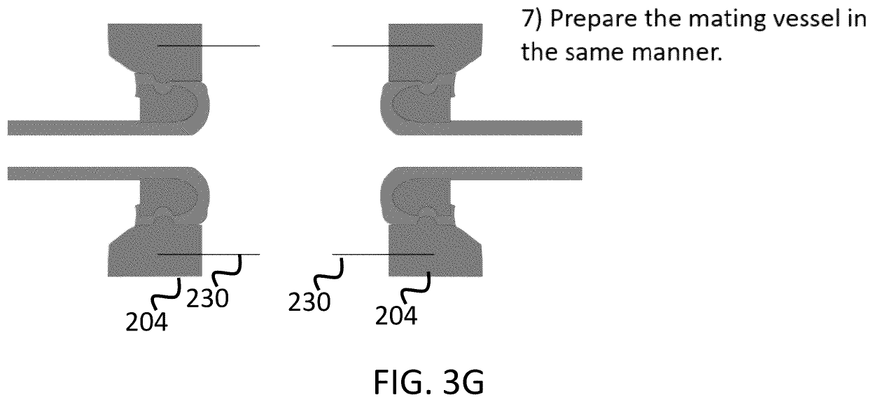

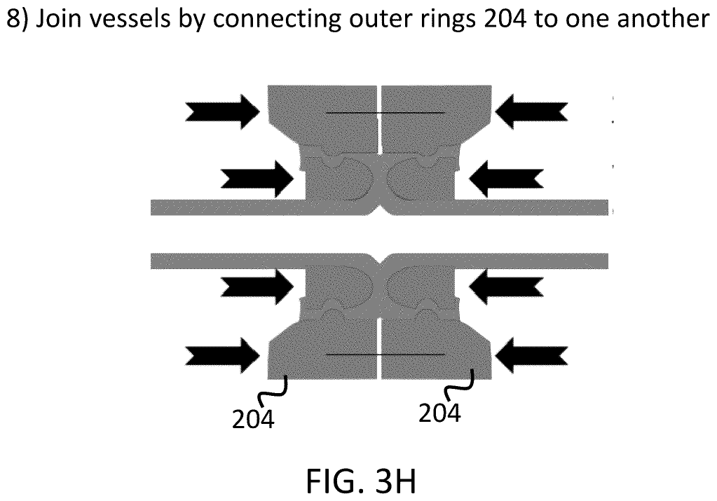

FIGS. 3A-3H show a preferred method for surgically installing the coupling device 200 on a vessel. In FIG. 3A the mating vessels 202 (donor vessel and recipient vessel) are prepared. The preparation for each is the same, so only one vessel 202 is shown. Preparation includes ensuring that both have adequate length and minimal tension for the anastomosis. Vessel blood flow is then clamped, and ischemia time begins. FIG. 3B shows that, the inner support ring 208 is slid over the outside of the vessel. The inner (lumen) wall of the artery remains undisturbed.

FIG. 3C shows the insertion of the support 212 into the vessel 202, which can be a temporary stenting guide tool into the lumen similar to a removable balloon catheter device with a supporting guide wire 214. FIG. 3D shows preparation of the eversion flaring guide tool 216, which is pre-connected to the outer ring 204. The outer ring 204 in this embodiment has pins 230 facing outward, which will serve as a connection interface 220 to the other outer ring 204.

FIG. 3E shows that the eversion flaring guide 216 is slid on the guide wire up to the support 212, which everts the end of the vessel 202. FIGS. 3E and 3F show that as the tapered surface 218 continues into the vessel lumen, eversion of the vessel end continues until the vessel wall is satisfactorily everted circumferentially, and the vessel wall protrudes past the inner ring. Then, such as with a locking tool device, the outer ring 204 is advanced in FIG. 3F until it seats and locks in place with the inner ring 208, capturing the everted portion of the vessel wall between the inner 208 and outer rings 204. The support 212 positioned to help prevent compression of the vessel 202 as the outer ring 204 is fit over the everted vessel portion and the inner ring 208. Once adequate capture of the vessel wall between the two rings is confirmed, the stenting tool (with the attached eversion flaring guide) is removed.

FIG. 3G shows that the same steps of FIGS. 3A-3F are used to prepare the mating (donor) vessel in the same manner as a recipient vessel, at which point the respective pins 230 (or other connection interfaces mentioned above), can be used in FIG. 3G, such as with a locking tool device, to join the host and the donor vessels by mating the outer rings can use using the pin capture mechanism 230. In FIG. 3H, anastomosis is complete. Clamps are removed and the physician ensures blood flow from donor vessel to recipient vessel. The coupling device is completely abluminal with respect to the two vessels after completing anastomosis.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A vascular anastomotic coupling device, comprising for each of two vessels prepared for anastomosis:
an inner ring sized to slide over an outer diameter of one of the two vessels;
an outer ring configured to trap an everted portion of the one of the two vessels between itself and the inner ring;
interface features on the inner ring and outer ring configured to trap and hold the everted portion of the one of the two vessels between them and fix a position of the inner ring and outer ring with respect to each other; and
a connection interface on the outer ring is configured, when mounted on the one of the two vessels, to join with the outer ring of the other of the two vessels to join the two vessels and complete anastomosis of the two vessels.

2. The vascular anastomotic coupling device of claim 1, comprising no feature that penetrates the two vessels after complete anastomosis.

3. The vascular anastomotic coupling device of claim 1, configured to be completely abluminal with respect to the two vessels after complete anastomosis.

4. The vascular anastomotic coupling device of claim 1, wherein the interface features comprise a circular groove and complementary ridge.

5. The vascular anastomotic coupling device of claim 4, wherein at least a portion of one of the interface features is flexible.

6. The vascular anastomotic coupling device of claim 4, wherein at least the interface feature on the outer ring is flexible.

7. The vascular anastomotic coupling device of claim 1, wherein at least the interface feature of the outer ring is flexible to expand slightly when it traps the everted portion between it and the inner ring.

8. The vascular anastomotic coupling device of claim 1, wherein the interface features comprise features of an inner circumference of the outer ring and an outer circumference of the inner ring.

9. The vascular anastomotic coupling device of claim 1, wherein the outer ring for each of the two vessels comprises a connection interface to interface with each other.

10. The vascular anastomotic coupling device of claim 9, wherein the connection interface comprises outward facing pins.

11. The vascular anastomotic coupling device of claim 9, wherein the connection interface comprises magnets, a mechanical capture, adhesive or suture openings.

12. The vascular anastomotic coupling device of claim 1, wherein the inner and outer rings are formed from flexible biocompatible material.

13. The vascular anastomotic coupling device of claim 1, wherein one or both of the inner and outer ring are formed from a biocompatible metal or alloy.

14. The vascular anastomotic coupling device of claim 13, wherein the metal or alloy is selected from the group consisting of stainless steel, cobalt-chrome alloy, titanium, nickel-titanium alloy (nitinol), platinum, silver, iridium, and tungsten.

15. A surgical kit comprising a plurality of different diameter sets of inner and outer rings of claim 1.

16. An anastomosis implantation device for implantation of the vascular anastomotic coupling device of claim 1, wherein the implantation device comprises:
a vessel support mounted on a guide wire and sized to fit within the vessel; and
a flaring tool configured to slide along the wire guide, carry an outer ring, and flare a portion of the vessel such that the outer ring can evert the portion over the inner ring.

17. The anastomosis implantation device of claim 16, wherein the flaring tool comprises a tapered, conical end to flare the portion of the vessel and a lumen to ride along the guide wire.

18. A surgical kit comprising anastomosis implantation device of claim 17 and a plurality of different diameter sets of inner and outer rings comprising for each of two vessels prepared for anastomosis:
an inner ring sized to slide over an outer diameter of one of the two vessels;
an outer ring configured to trap an everted portion of the one of the two vessels between itself and the inner ring;
interface features on the inner ring and outer ring configured to trap and hold the everted portion of the one of the two vessels between them and fix a position of the inner ring and outer ring with respect to each other; and
a connection interface on the outer ring is configured, when mounted on the one of the two vessels, to join with the outer ring of the other of the two vessels to join the two vessels and complete anastomosis of the two vessels.
